(19) United States
(12) Patent Application Publication
(10) Pub. No.: US 2022/0285016 A1
(43) Pub. Date: Sep. 8, 2022

(54) MANAGEMENT SYSTEM, INFORMATION PROCESSING APPARATUS, AND COMPUTER READABLE MEDIUM STORING PROGRAM FOR MANAGING RECORDS OF PROCESSING

(71) Applicant: CANON KABUSHIKI KAISHA, Tokyo (JP)

(72) Inventor: Motohiro Furusawa, Shizuoka (JP)

(21) Appl. No.: 17/675,242

(22) Filed: Feb. 18, 2022

(30) Foreign Application Priority Data
Mar. 8, 2021 (JP) 2021-036676

(51) Int. Cl. G16H 40/20; G06Q 10/06

(52) U.S. Cl. CPC G16H 40/20; G06Q 10/0633

(57) ABSTRACT

According to an aspect of the present disclosure, a management system is operable to manage a workflow including a plurality of processes for executing predetermined processing on a processing target. The workflow includes a first process and a second process executed subsequently to the first process. The management system includes one or more processors and one or more memories storing one or more programs. The one or more programs, when executed by the one or more processors, cause the one or more processors to perform generation processing in which information for determining a temporal transition of a number of processing targets for which the first process has been completed but the second process has not been started or completed is generated based on management information stored in one or more storage devices.

### BACKGROUND OF THE INVENTION

### Field of the Invention

The present invention relates to a technique for managing and utilizing records of processing.

### Description of the Related Art

Sterilization targets such as medical instruments are sterilized at hospitals, for example. At this time, in order to determine an achievement degree of sterilization for a sterilization target, a so-called chemical indicator (hereinafter, CI) for sterilization is used. A CI has a discoloration area which changes color according to the achievement degree of a condition necessary for sterilization by a sterilizing agent (vapor, hydrogen peroxide, or the like). Japanese Patent No. 3414291 discloses a management apparatus for managing sterilization records. According to Japanese Patent No. 3414291, the management apparatus includes determination results by CI in sterilization records.

Also, medical instruments are cleaned after use at hospitals. At this time, a cleaning indicator is used to determine the achievement degree of cleaning. A cleaning indicator has a discoloration area which changes color depending on the achievement degree of a condition necessary for cleaning by a cleaning agent.

In recent years, importance has been placed on keeping the quality of sterilization and cleaning high. Japanese Patent No. 3414291 discloses including determination results by CI in sterilization records; but does not disclose providing information for improving the quality of sterilization based on the sterilization records.

### SUMMARY OF THE INVENTION

The present invention provides a management system, an information processing apparatus, and a program that can provide information for improving the quality of predetermined processing.

According to an aspect of the present disclosure, a management system operable to manage a workflow including a plurality of processes for executing predetermined processing on a processing target is provided. Here, the workflow includes a first process and a second process executed subsequently to the first process. The management system comprising: one or more processors; one or more memories configured to store one or more programs; and one or more storage devices configured to store, management information including information, for each of processing targets, indicating a completion timing of the first process and a start timing or a completion timing of the second process, wherein the one or more programs, when executed by the one or more processors, cause the one or more processors to: perform generation processing in which information for determining a temporal transition of a number of processing targets for which the first process has been completed but the second process has not been started or a number of processing targets for which the first process has been completed but the second process has not been completed is generated based on the management information.

Further features of the present invention will become apparent from the following description of exemplary embodiments with reference to the attached drawings.

### BRIEF DESCRIPTION OF THE DRAWINGS

FIG. 1 is a view illustrating a sterilization workflow according to an embodiment.

FIG. 2 is a configuration diagram of a management system according to an embodiment.

FIG. 3 is a view illustrating a CI according to an embodiment.

FIG. 4 is a configuration diagram of a measuring apparatus according to an embodiment.

FIG. 5 is a view illustrating a portion of management information according to an embodiment.

FIG. 6 is a view illustrating a portion of management information according to an embodiment.

FIG. 7 is a view illustrating information that an information processing apparatus provides, according to an embodiment.

FIG. 8 is a view illustrating information that the information processing apparatus provides, according to an embodiment.

FIG. 9 is a view illustrating information that the information processing apparatus provides, according to an embodiment.

FIG. 10 is a view illustrating information that the information processing apparatus provides, according to an embodiment.

FIG. 11 is a view illustrating information that the information processing apparatus provides, according to an embodiment.

FIG. 12 is a view illustrating information that the information processing apparatus provides, according to an embodiment.

FIG. 13 is a functional block diagram of the information processing apparatus according to an embodiment.

FIG. 14 is a functional block diagram of the management system according to an embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

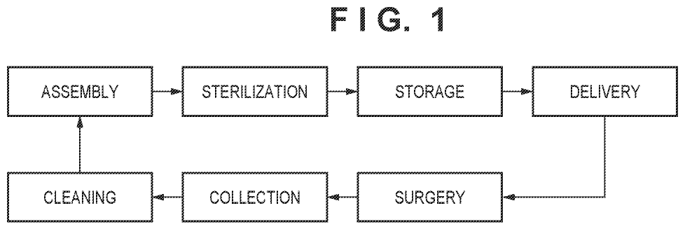

FIG. 1 illustrates a sterilization workflow. In this embodiment, a sterilization workflow means a series of processes performed for sterilization of a sterilization target. An assembly process is a process of grouping and packaging one or more sterilization targets. Note that, in some cases, the number of sterilization targets included in one group may be one. Groups may be made according to a correspondence between surgery in which the sterilization targets are used and sterilization apparatuses by which the sterilization targets are sterilized and the like. For example, sterilization targets used in the same surgery and sterilized by the same sterilization apparatus may be grouped into the same group. In the assembly process, one or more sterilization targets of one group and a CI (first indicator) are packaged in one packaging member. This one packaging member is also called one set in the following. One set is also one sterilization target. A plurality of the same sterilization targets are used at hospitals and the like, so a plurality of sets including the same sterilization targets is formed. Hereinafter, a set including the same sterilization targets will be referred to as a set of the same type. Conversely, when two sets differ in type, it means that at least one of the sterilization targets included in the two sets is different. A CI to be packaged in a set in the assembly process is determined, for example, according to the type of sterilization apparatus that sterilizes the set.

A sterilization process is a process of sterilizing a set by a sterilization apparatus. Note that one sterilization apparatus can sterilize a plurality of sets at the same time. The achievement degree of sterilization for each set is determined based on the CI enclosed in that set. However, after sterilization by the sterilization apparatus, each set is stored as packaged; the package is opened and the sterilization target and the CI in the package are taken out immediately before using the set in surgery or the like. For this reason, when performing sterilization for one or more sets by a sterilization apparatus, a process challenge device (PCD) in which a CI (second indicator) is enclosed is also placed inside the sterilization apparatus. Note that the PCD is placed at a predetermined position in the sterilization apparatus, for example, at a position where the achievement degree of sterilization is likely to be lower in the sterilization apparatus. After sterilization by the sterilization apparatus, the achievement degree of sterilization for one or more sets by the sterilization apparatus is determined by the discoloration area of the CI placed in the PCD. When the achievement degree of sterilization determined by the CI placed in the PCD is equal to or higher than a quality threshold, all sets sterilized by the sterilization apparatus are transferred to a subsequent storage process. On the other hand, when the achievement degree is less than the quality threshold, all of the sets sterilized by the sterilization apparatus are transferred to the assembly process again. In the following description, the achievement degree of sterilization determined by the CI of a PCD placed in a sterilization apparatus together with one or more sets is also referred to as a “PCD result” for the one or more sets or a “PCD result” for the sterilization apparatus.

A storage process is a process in which sets sterilized by the sterilization apparatus are moved to a storage cabinet to be stored. As described above, the sets are stored packaged.

A delivery process is a process for moving the sets to be used in surgery or the like from a storage cabinet to an operation room or the like where they are used.

A surgical process is also a utilization process in which sterilization targets are used. In the surgical process, first, the package of a set is opened and the sterilization targets and the CI therein are taken out. Then, based on the color of the discoloration area of the CI that has been taken out, the achievement degree of sterilization for the set is determined. If the achievement degree of sterilization is equal to or higher than the quality threshold, the sterilization targets of the set are actually used in surgery or the like. On the other hand, when the achievement degree of sterilization is less than the quality threshold, the sterilization targets of the set are not used and are transferred to a subsequent collection process.

A collection process is a process of moving, to where a cleaning process is performed, sterilization targets used in the surgical process or sterilization targets not used due to the achievement degree of sterilization determined in the surgical process being less than the quality threshold.

A cleaning process is a process of cleaning sterilization targets collected in the collection process. After the set has been cleaned in the cleaning process, the set is transferred again to the assembly process.

Note that the sterilization workflow described above is only one example, and the sterilization workflow of the present invention is not limited to the above processes. For example, the sterilization workflow may include processes other than the above processes. Further, the sterilization workflow may be one in which at least one process of each of the above processes is further divided into a plurality of processes. Further, the sterilization workflow may be one in which at least two processes of the above processes are further combined into one process.

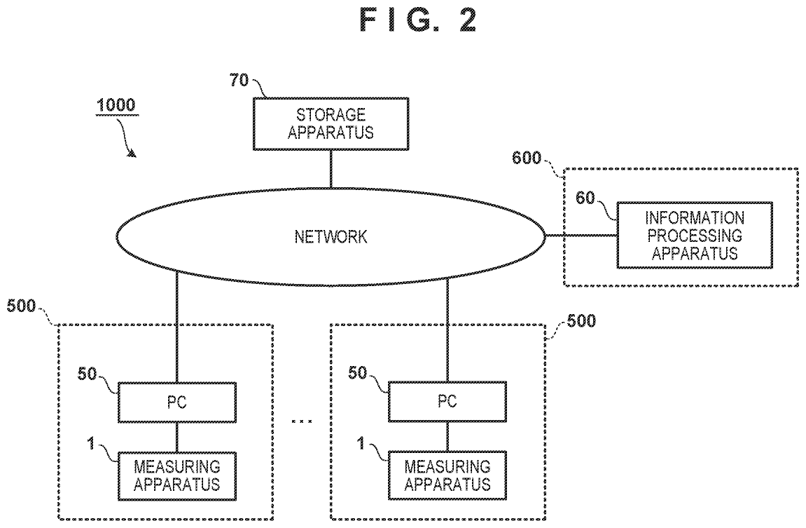

FIG. 2 is a configuration diagram of a management system 1000 for managing the above sterilization workflow. In FIG. 2, reference numerals 500 denote facilities, such as hospitals, that use sterilization targets, and a reference numeral 600 denotes a management company that monitors the sterilization workflow of each facility. In the following explanation, those performing the work related to sterilization at each facility are denoted as “workers”, and those managing and supervising the sterilization workflow at each facility are denoted as “managers”. In addition, those monitoring the sterilization workflow of each facility at the management company 600 and presenting information to each facility are referred to as “monitors”. The management system 1000 includes personal computers (hereinafter, PCs) 50, a storage apparatus 70 and an information processing apparatus 60 that can communicate with each other via a network.

Each facility 500 has a PC 50 which is connected to the network. At each facility 500, a worker inputs information to be described later into the PC 50 in each process of the sterilization workflow. Note that, since each process of the sterilization workflow may be performed at various locations in the facility, a PC 50 may be provided where each process of the sterilization workflow is performed. That is, the number of PCs 50 that each facility 500 has is not limited to one, and each facility 500 may have a plurality of PCs 50 which connects to the network. Also, some of the information is inputted to a PC 50 via a measuring apparatus 1. Thus, a measuring apparatus 1 can be connected to a PC 50 to which information needs to be inputted from the measuring apparatus 1. The PC 50 transmits the inputted information to the storage apparatus 70 via the network along with facility specification information for specifying the facility 500. Note that a PC 50 of a facility can access the information of the facility stored in the storage apparatus 70.

The storage apparatus 70 stores information received from each facility 500 in association with the facility specification information of corresponding facility 500. In the following description, information stored in the storage apparatus 70 is referred to as management information. In the present embodiment, the storage apparatus 70 is a function for storing information provided by a so-called cloud service. Note that the storage apparatus 70 need not be a single apparatus and may be a plurality of geographically-dispersed storage apparatuses.

The information processing apparatus 60 of the management company 600 is, for example, a computer, and can access and acquire (download) the management information held by the storage apparatus 70 via the network. Further, the information processing apparatus 60 can present, to a monitor, information to be described later based on the management information. Note that, in the present embodiment, presenting information to a monitor includes not only displaying the information on a display of the information processing apparatus 60 but also storing the information in a storage device of the information processing apparatus 60. In addition, a monitor may operate the information processing apparatus 60 to transmit the presented information to a PC 50 of a facility 500.

In the present embodiment, each set is given identification information. Identification information can be in the form of a serial number or barcode printed on a sticker, for example. Alternatively, identification information can be recorded in an IC tag. The medium having the identification information is adhered to an outer surface of a packaging member of a set or to a sterilization target of a set, for example. Alternatively, it can be adhered to a CI enclosed in a set. In addition, if a serial number or the like is recorded on a CI itself, the serial number of the CI enclosed in a set can be used as the identification information of that set. In this embodiment, it is assumed that the identification information of a barcode format is adhered to the outer surface of the packaging member of a set. Incidentally, this identification information is associated with the serial number of the CI enclosed in the same set. That is, the serial number of the CI and the identification information adhered to the outer surface of the packaging member have a one-to-one relationship. Therefore, the serial number of a CI is also the identification information of a set.

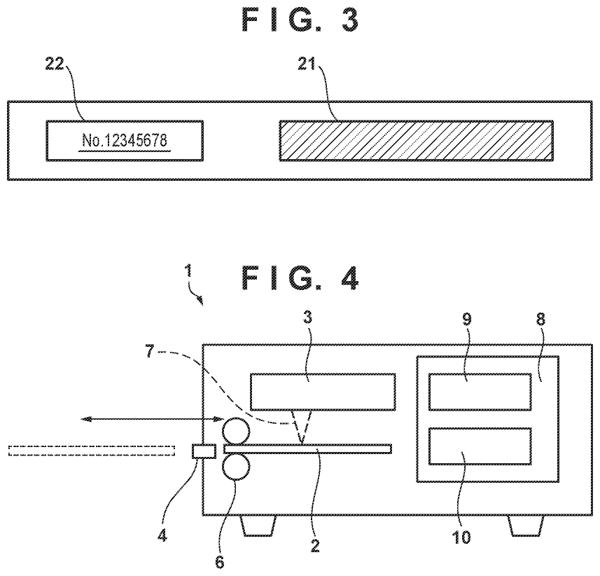

FIG. 3 illustrates a CI according to the present embodiment. A CI is a sheet-like test paper and has a discoloration area 21, which has been chemically treated, on its surface. The color of the discoloration area 21 changes depending on the achievement degree of sterilization. For example, the discoloration area 21 of the CI is a color value C#1 in an initial state and is configured so as to change from a color value C#2 to a color value C#9 according to the achievement degree of sterilization, and finally become constant at a color value C#10. The CI also includes an identification portion 22 which indicates a serial number. Further, in the present embodiment, it is assumed that the type of CI can be determined from the serial number. For example, the predetermined number of digits at the beginning of the serial number indicates the type of CI.

FIG. 4 is a cross-sectional view illustrating an example of a measuring apparatus 1. Incidentally, a reference numeral 2 in FIG. 4 indicates a CI that has been conveyed to the measurement position of the measuring apparatus 1, and the dotted lines indicate the CI before being inserted into the measuring apparatus 1. The CI that has been inserted into an insertion portion 4 of the measuring apparatus 1 is conveyed to the measurement position by a pair of conveying rollers 6. A measuring unit 3 measures the color of the discoloration area 21 of the CI. After the CI has been measured, the CI is discharged to the outside of the measuring apparatus 1 by rotating the pair of conveying rollers 6 in reverse. The measuring unit 3 reads an optical measurement value, for example, a spectral reflectance, associated with the color value of the discoloration area 21 of the CI using light 7. A control unit 8 controls the measuring apparatus 1. Various information used for measurement is stored in a storage unit 9 of the control unit 8. A calculation processing unit 10 of the control unit 8 determines the color value of the discoloration area 21 of the CI based on the result of measurement, for example, the spectral reflectance, by the measuring unit 3. The control unit 8 outputs the color information indicating the determined color value to a PC 50. It is noted that the measuring apparatus 1 is not limited to those for determining the color value of the discoloration area 21 by reading the spectral reflectance. For example, the measuring apparatus 1 may read the color value of the discoloration area 21 by capturing the discoloration area 21 with an image sensor.

Incidentally, although the measuring apparatus 1 illustrated in FIG. 4 discharges a CI from the insertion portion 4, it may be provided with a discharge portion on the side opposite to the insertion portion 4 and configured to discharge the CI from the discharge portion after measurement. Further, although the measuring apparatus 1 illustrated in FIG. 4 measures a CI with the fixed measuring unit 3 after conveying the CI to the measurement position, the measuring unit 3 may configured to be movable. That is, it is also possible to have a configuration in which the discoloration area 21 of the CI is read by moving the measuring unit 3 in a predetermined direction. Further, a configuration may be taken such that a worker moves the measuring apparatus 1 over a CI to read the discoloration area 21 of the CI like a barcode reader. Such a configuration is advantageous when utilizing CIs of various sizes.

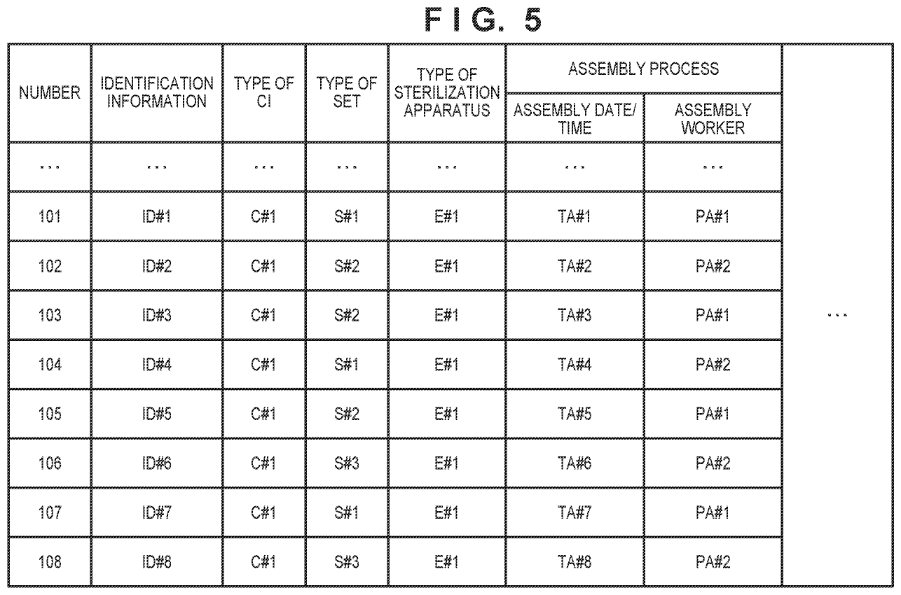

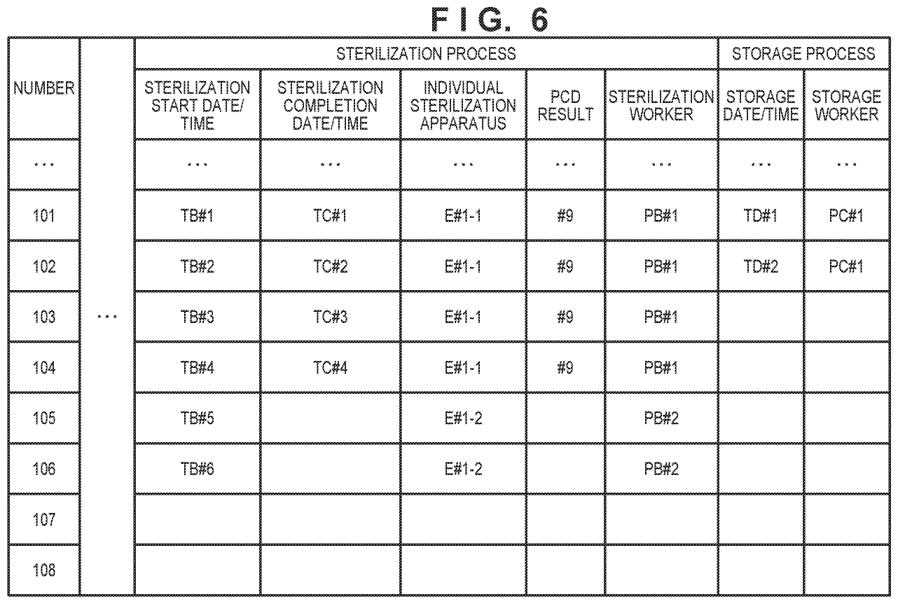

FIGS. 5 and 6, in the present embodiment, illustrate a portion of the management information that the storage apparatus 70 stores and holds. It is noted that FIGS. 5 and 6 illustrate a portion of the management information relating to a single given facility. A number field indicates the number of each record. Each record is provided for identification information of each set. An identification information field contains the identification information of a set. In this embodiment, identification information of a set is information in a barcode format adhered to that set and is associated with the serial number of a CI enclosed in that set. A CI type field contains information indicating the type of CI specified from the serial number of a CI enclosed in that set. A set type field contains information that specifying the type of that set. A sterilization apparatus type field contains information indicating the type of sterilization apparatus to be sterilizing that set.

An assembly date/time field stores information indicating the date and time the assembly process for that set has been completed (hereinafter, completion timing). An assembly worker field stores information specifying an assembly worker who performed the assembly and packaging of that set.

Proceeding to FIG. 6, a sterilization start date/time field stores information indicating the date and time for when that set was placed in the sterilization apparatus (hereinafter, start timing). A sterilization completion date/time field stores information indicating the date and time for when that set has been sterilized by the sterilization apparatus and then taken out from the sterilization apparatus (hereinafter, completion timing). An individual sterilization apparatus field contains information indicating an individual sterilization apparatus that sterilized that set. A PCD result field stores a PCD result, that is, information indicating the achievement degree of sterilization determined based on a CI in a PCD placed in the sterilization apparatus together with that set. A sterilization worker field stores information specifying a sterilization worker who performed the sterilization of that set.

A storage date/time field stores information indicating the date and time the storage process for that set has been completed (hereinafter, completion timing). A storage worker field stores information specifying a storage worker who performed the storage of that set.

Note that, in this example, it is assumed that a worker holds a card in which specification information specifying the worker is recorded. In this example, it is assumed that the specification information of the worker is also in a barcode format.

For example, when an assembly worker begins forming a single set in the assembly process, the assembly worker inputs to a PC 50 that the process is currently the assembly process. Then, the assembly worker inputs specification information of the assembly worker into the PC 50 by reading his/her specification information with a barcode reader of the PC 50. Subsequently, the assembly worker inputs information indicating the type of set that he/she assembled. The PC 50 presents, to the assembly worker, information indicating the type of CI to be enclosed in the set based on the type of the set inputted. The assembly worker selects a CI to be enclosed in the set from the CIs of the type presented, inputs its serial number into the PC 50, and encloses it in the set. Incidentally, at this time, the PC 50 determines that the type to be determined from the inputted serial number is the same as the type presented and issues a warning to the assembly worker if not the same. Subsequently, the assembly worker adheres the identification information to the packaging member and reads the adhered identification information (identification information of the set) with the barcode reader of the PC 50. When the PC 50 reads the identification information of the set, it verifies that the management information does not contain a record of that identification information. If the management information includes a record of the identification information, the PC 50 outputs a warning that the identification information of the set is repetitive and prompts the assembly worker to change the identification information.

If the management information does not include a record of the identification information, the PC 50 adds a record of the identification information to the management information. Then, the PC 50 records the read identification information in the identification information field and records the read specification information in the assembly worker field. In addition, the PC 50 records information indicating the type of inputted set in the set type field and records information indicating the type of enclosed CI in the CI type field. In addition, the PC 50 records, in the sterilization apparatus type field, information indicating the type of sterilization apparatus, which has been determined based on the type of set, to perform sterilization of the set of that type. Although not specified in FIG. 5, the serial number of the CI shall also be recorded. Finally, the PC 50 records, in the assembly date/time field, the completion timing at which all inputs have been completed. Records with information recorded in all of the fields as clearly illustrated in FIG. 5 indicate that the assembly process has been completed for the sets corresponding to those records.

Subsequently, when starting sterilization, a sterilization worker inputs into the PC 50 that the reading is being performed prior to sterilization and then reads the identification information of the set to be sterilized and the specification information of the sterilization worker with a barcode reader. The PC 50 searches for a record of the read identification information. If there is no record of the read identification information, or if there is a record of the read identification information but not all of the information that needs to be inputted in the process one upstream has been recorded, the PC 50 displays a warning to the worker. When there is a record of the read identification information and the process one upstream has been completed, the PC 50 records the timing of the reading as the start timing in the sterilization start date/time field of the found record. In addition, the PC 50 records the read specification information in the sterilization worker field of the found record. The sterilization worker places the set from which the identification information has been read in the sterilization apparatus. In addition, the worker records information specifying an individual sterilization apparatus in which the set has been placed in the individual sterilization apparatus field of the record corresponding to that set.

When removing the set from the sterilization apparatus after the sterilization apparatus has completed sterilization, the sterilization worker reads the identification information of that set with the barcode reader of the PC 50. Note that the reading is performed after it is inputted in advance into the PC 50 that the reading is being performed after sterilization. The PC 50 searches for the record of the read identification information and records the timing of the reading as the completion timing in the sterilization completion date/time field of the found record. Further, the sterilization worker measures, with the measuring apparatus 1, the discoloration area 21 of the CI of the PCD placed in the sterilization apparatus together with that set. The PC 50 determines the achievement degree of sterilization based on the color value of the discoloration area 21 acquired from the measuring apparatus 1 and records the determined achievement degree in the PCD result field. Note that, in this embodiment, when the color value of the discoloration area 21 is C#m to C#m+1 (m is an integer from 1 to 9), it is assumed that the achievement degree of sterilization is #m. Note that the larger the value of m, the higher the achievement degree of sterilization.

For a record for which the sterilization process has been completed and accordingly information has been recorded in all the fields relating to the sterilization process, the PC 50, for example, prints a report indicating the completion of the sterilization process. The report may be made to include one or more or all of the information recorded in the sterilization process-related fields. That this print output has been performed is a condition for proceeding to the subsequent storage process. The same can be applied to transitions between other processes.

From FIGS. 5 and 6, it can be seen that the sterilization process has been completed for the four sets corresponding to the records of numbers 101 to 104, because information has been set in the sterilization completion date/time field and the PCD result field. Incidentally, in FIG. 6, the start timings recorded in the sterilization start date/time fields of the records having the numbers 101 to 104 are substantially the same, and the completion timings recorded in the sterilization completion date/time fields are also substantially the same. Since the start timings and the completion timings of the sterilization of the records having the number 101 to 104 are respectively substantially the same, and the numbers 101 to 104 have been sterilized with the same individual sterilization apparatus, the information processing apparatus 60 can determine that these 4 sets have been sterilized by an individual sterilization apparatus E #1-1 at the same time. Note that information explicitly indicating sets which have been sterilized by a single sterilization apparatus at the same time can also be added to the management information.

In addition, for the two sets corresponding to the records of numbers 105 and 106, the start timings have been set in the sterilization start date/time fields, but the completion timings have not been set in the sterilization completion fields. Thus, it can be seen that the 2 sets corresponding to the records of the numbers 105 and 106 are in a state in which sterilization by the sterilization apparatus is in progress or sterilization by the sterilization apparatus has been completed but the set has not been taken out. In addition, it can be seen that the two sets corresponding to the records of numbers 107 and 108 are waiting to transition from the assembly process to the sterilization process, because the start timings have not been set in the sterilization start date/time fields. Note that, in this example, it is assumed that a single worker performs sterilization of one or more sets by a given sterilization apparatus and performs the determination of PCD results. Therefore, only one sterilization worker is indicated in the sterilization worker field. However, when a worker who places a set and a PCD in a sterilization apparatus to start sterilization, a worker who takes out a set from a sterilization apparatus after sterilization has been completed, and a worker who determines a PCD result can be different from each other, a configuration is taken so as to provide a field indicating each sterilization worker.

After the sterilization process has been completed, the storage worker moves the sets for which sterilization has been completed to a storage cabinet. Then, after inputting in the PC 50 that the reading is at the time of storage, the storage worker reads the identification information of the sets to be stored and his/her specification information with a barcode reader. The PC 50 searches for the record of the read identification information and records the timing of the reading as the completion timing in the storage date/time field of the found record. In addition, the PC 50 records the read specification information in the storage worker field of the found record.

From FIGS. 5 and 6, it can be seen that the storage process has been completed for the two sets corresponding to the records of the numbers 101 and 102, because information has been set in the storage date/time field. Meanwhile, it can be seen that the storage process has not been completed for the two sets corresponding to the records of the numbers 103 and 104, because information has not been set in the storage date/time field. More particularly, it can be seen that the two sets are in a state either of waiting to transition from the sterilization process to the storage process or in which a storage operation is in progress.

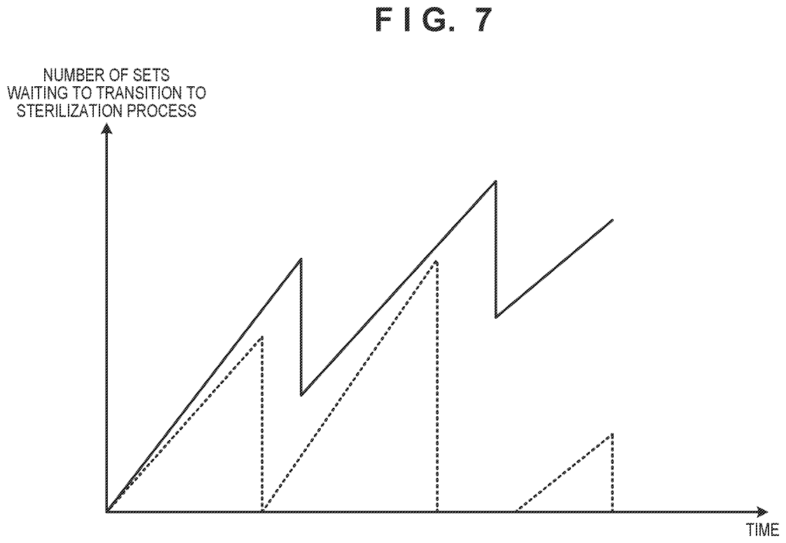

FIG. 7 illustrates an example of information that can be generated by the information processing apparatus 60 from the management information illustrated in FIGS. 5 and 6. As described above, the information processing apparatus 60 can determine, from the management information illustrated in FIGS. 5 and 6, the number of sets waiting to transition to the sterilization process at a given timing. Therefore, as illustrated in FIG. 7, the information processing apparatus 60 can present, to a monitor, information illustrating the temporal transition of the number of sets waiting to transition to the sterilization process for each facility. For example, in the graph of FIG. 7, a point in time at which the number of sets waiting to transition to the sterilization process is decreasing indicates that one or more sets have been placed in the sterilization apparatus due to the sterilization process starting. As illustrated by a solid line in FIG. 7, the number of sets waiting to transition to the sterilization process not becoming a sufficiently smaller number at this timing of reduction suggests that the number of sterilization apparatuses may be insufficient. In addition, as illustrated by a dotted line in FIG. 7, the number of sets waiting to transition to the sterilization process always becoming 0 due to the sterilization process starting and that number continuing to be 0 over a time suggests that there is a possibility that the number of sterilization apparatuses may be excessive. Therefore, by a monitor providing the information illustrated in FIG. 7 to each facility, a manager of each facility can devise measures to optimize the number of sterilization apparatuses. Thus, the quality of sterilization can be improved.

It is noted that, in this example, the temporal transition of the number of sets waiting to transition to the sterilization process has been taken as an example; however, it is also possible to display the temporal transition of the number of sets waiting to transition to another process. For example, it can be seen that the temporal transition of the number of sets for which the sterilization process has been completed but the storage process has not been completed can be presented from the management information of FIGS. 5 and 6. The number of sets for which the storage process has not been completed decreases by the storage worker moving one or more sets to a storage cabinet and storing them therein. It is possible to determine, from the temporal transition of the number of sets for which the storage process has not been completed, whether the number of storage workers is appropriate or whether the number of sets to be moved to a storage cabinet at a time is appropriate, for example. Note that if it is desired to distinguish between the number of sets waiting to transition to the storage process and the number of sets for which storage operation is in progress among the number of sets for which the storage process has not been completed, configuration may be taken so as to record the start timing of the storage operation and the completion timing of the storage operation in the management information. The same applies to transitions between other processes.

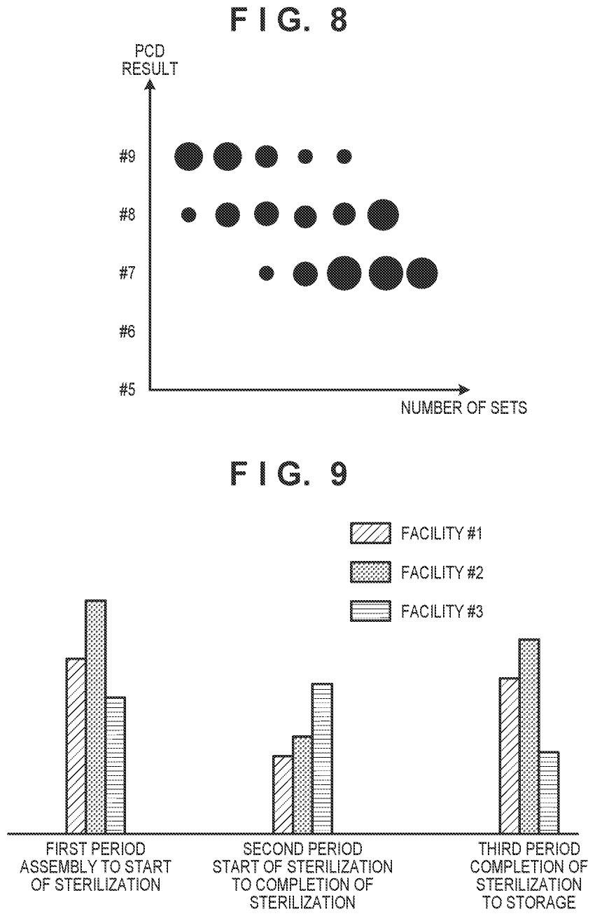

FIG. 8 illustrates another example of information that can be generated by the information processing apparatus 60 from the management information illustrated in FIGS. 5 and 6. As described above, it is possible to determine, from the management information illustrated in FIGS. 5 and 6, the number of sets sterilized by a single given sterilization apparatus and the PCD results of those sterilizations. The graph in FIG. 8 illustrates the relationship between the number of sets sterilized with a single sterilization apparatus and the PCD results. Note that the size of a circle indicates the number of times that achievement degree has been obtained. More specifically, the greater the size of a circle, the greater the number of times that achievement degree has been obtained. From FIG. 8, it can be seen that PCD results tend to worsen when the number of sets to be sterilized by a single sterilization apparatus is increased. Note that the graph of FIG. 8 may be configured, for example, to be presented for each individual sterilization apparatus or for each type of sterilization apparatus. For example, when the graph of FIG. 8 is presented for each individual sterilization apparatus, a manager of each facility can determine an upper limit value of the number of sets to be sterilized by those individual sterilization apparatus. In addition, when the graph of FIG. 8 is presented for each type of sterilization apparatus, the manager of each facility can determine, from that graph, an upper limit value of the number of sets to be sterilized by those types of sterilization apparatus. In addition, in either case, it becomes possible to determine the number of sterilization apparatuses necessary for the number of sets to be sterilized by a sterilization apparatus to be equal to or lower than the determined upper limit value. Thus, the quality of sterilization can be improved.

Further, the information processing apparatus 60 can also present information comparing, between the facilities, the relationship between the number of sets sterilized in a single sterilization apparatus and the PCD results. For example, assume that the graph of FIG. 8 is created for each type of sterilization apparatus. At this time, both the results of each facility on its own and results in which all facilities have been consolidated are created and compared. For example, it is possible to determine whether there are facilities in which the degree of degradation of PCD results due to an increase in the number of sets sterilized by a single sterilization apparatus is higher than the result in which all facilities have been consolidated. If such facilities exist, that facilities may have a problem with the method of use of sterilization apparatuses or the sterilization apparatuses in use may have degraded over time. Therefore, the monitor can inform such facilities that there may be a problem with the method of use of sterilization apparatuses or a problem with the sterilization apparatuses themselves.

FIG. 9 illustrates another example of information that can be generated by the information processing apparatus 60 from the management information illustrated in FIGS. 5 and 6. The information processing apparatus 60 can determine a period (hereinafter, first period) until the start of sterilization after completion of the assembly process based on the completion timing recorded in the assembly date/time field and the start timing recorded in the sterilization start date/time field of the management information. Also, the information processing apparatus 60 can determine a period (hereinafter, second period) from the start of sterilization until completion of sterilization based on the start timing recorded in the sterilization start date/time field and the completion timing recorded in the sterilization completion date/time field of the management information. Also, the information processing apparatus 60 can determine a period (hereinafter, third period) from completion of sterilization until completion of the storage process based on the completion timing recorded in the sterilization completion date/time field and the completion timing recorded in the storage date/time field of the management information.

The graph of FIG. 9 illustrates, for each facility, the sum of the first period, the sum of the second period, and the sum of the third period for one or more sets sterilized by one sterilization apparatus. From the graph of FIG. 9, it can be determined that facilities which have a period for which the sum is longer than the other facilities have a problem in the operations related to that period for which the sum is longer. Thus, for example, it is possible to devise measures such as increasing the number of workers or placing more experts. Note that, in FIG. 9, the sum of each period of one or more sets sterilized by one sterilization apparatus has been assumed; however, an average working time per set in a given period of time can be assumed as well. Specifically, by dividing the total number of the first period in a given period by the number of sets processed in that period, it is possible to determine the average work time per set. Furthermore, by presenting the work time per type of set, it is also possible to determine, for each type of set, a set for which it takes more time to do the work and devise measures for improving work efficiency for that set. Thus, the quality of sterilization can be improved.

Incidentally, although the first period, the second period, and the third period have been described as an example based on the timings specified in FIGS. 5 and 6, the present invention can be applied to other periods. For example, in the present embodiment, only the completion timing of the assembly process is recorded, however, by recording both the start timing and the completion timing of the assembly process, it becomes possible to determine the work time of the assembly process in a similar manner. The same applies to other processes not illustrated in FIGS. 5 and 6.

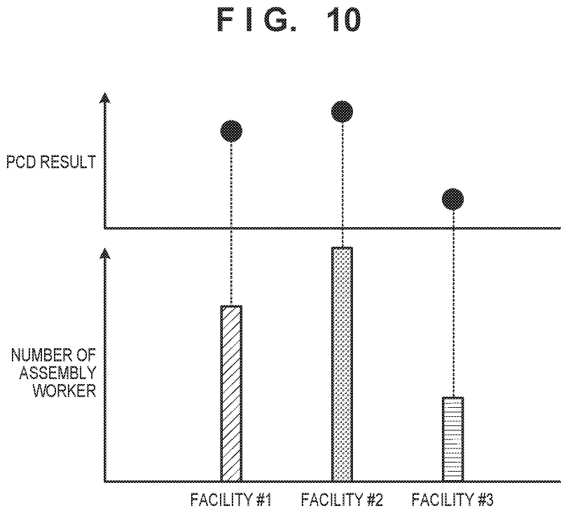

FIG. 10 illustrates another example of information that can be generated by the information processing apparatus 60 from the management information illustrated in FIGS. 5 and 6. The lower graph of FIG. 10 illustrates the number of assembly workers at each facility, and the upper graph of FIG. 10 illustrates the average value of the PCD results. For example, if the PCD results for a facility with fewer assembly workers, when compared to other facilities, are lower than that for other facilities, such as facility #3 in FIG. 10, the monitor may propose increasing the number of assembly workers to the manager of the facility. Thus, the quality of sterilization can be improved.

Incidentally, although, in FIG. 10, the numbers of assembly workers are compared, configuration may be taken so as to compare the number of workers of other processes such as the number of sterilization workers.

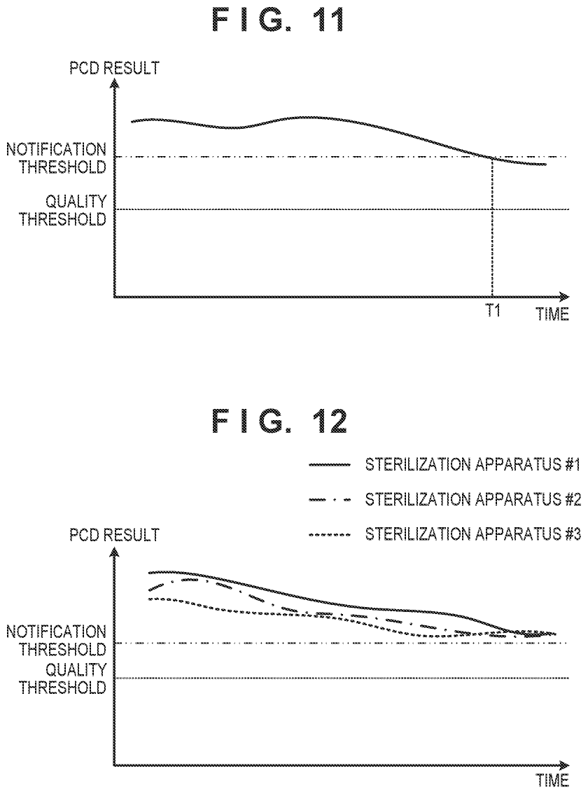

FIG. 11 illustrates another example of information that can be generated by the information processing apparatus 60 from the management information illustrated in FIGS. 5 and 6. Based on the individual sterilization apparatus field, the sterilization completion date/time field, and the PCD result field of the management information, the information processing apparatus 60 can determine the temporal transition of the achievement degree of sterilization by individual sterilization apparatuses. FIG. 11 illustrates the temporal transition of the achievement degree of sterilization by a given, single, individual sterilization apparatus. Note that the time on the horizontal axis uses the completion timing indicated by the sterilization completion date/time field. In FIG. 11, a quality threshold is a threshold for determining whether sterilization is OK or NG (not okay). When the PCD result is smaller than the quality threshold, the set sterilized by the sterilization apparatus is transferred again to the assembly process. Further, in FIG. 11, a notification threshold is a threshold for recommending maintenance of the sterilization apparatus. Incidentally, the notification threshold is set to a higher value of achievement degree than the quality threshold.

In FIG. 11, the PCD result from time T1 is lower than the notification threshold. The information processing apparatus 60 detects a sterilization apparatus in which the PCD results have fallen below the notification threshold and outputs information indicating the detected sterilization apparatus. This allows the monitor to recommend maintenance of the sterilization apparatus in which the PCD results have fallen below the notification threshold to the manager of a facility using that sterilization apparatus. Thus, it is possible to perform maintenance for the sterilization apparatus before the achievement degree of sterilization can remain lower than the quality threshold for an extended period due to deterioration of the sterilization apparatus with time or the like. Thus, the quality of sterilization can be improved.

FIG. 12 illustrates another example of information that can be generated by the information processing apparatus 60 from the management information illustrated in FIGS. 5 and 6. The information illustrated in FIG. 12 has been generated to enable a comparison of the temporal transition of the PCD results of the sterilization apparatus of the same facility between individual sterilization apparatuses. According to FIG. 12, none of sterilization apparatuses #1, #2, and #3 are below the notification threshold. However, the PCD results according to sterilization apparatuses #1, #2, and #3 have decreased similarly with time, and sterilization apparatuses #1, #2, and #3 may fall below the notification threshold in the same time period. If the sterilization apparatuses #1, #2, and #3 enter a maintenance state at the same time, there is a possibility that there may be a shortage of sterilization apparatuses that can be used in the sterilization process. Therefore, the information processing apparatus 60 presents the temporal transition of the PCD results of each sterilization apparatus as illustrated in FIG. 12, so that, for example, the monitor can propose to the manager to adjust the maintenance timing for the sterilization apparatuses #1, #2, and #3 so that they do not enter the maintenance state at the same time. For example, from the graph of FIG. 12, it can be recommended that the maintenance of the sterilization apparatus #3, which is not below the notification threshold, but whose PCD results have come close to the notification threshold earlier, be performed earlier. Thus, the quality of sterilization can be improved.

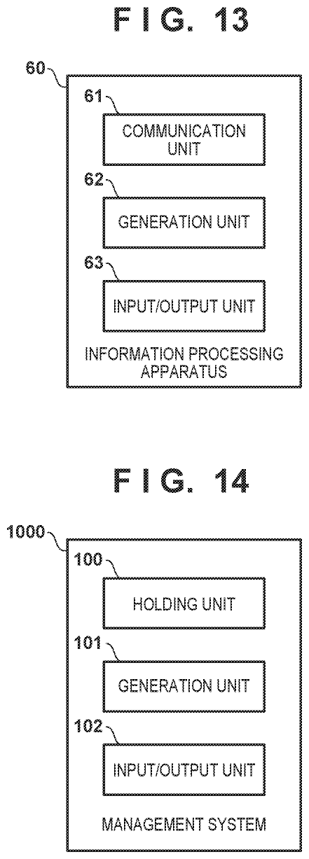

FIG. 13 is a functional block diagram of the information processing apparatus 60 according to the present embodiment. Note that the information processing apparatus 60 may be implemented by a computer having one or more processors, one or more memory devices, suitable input/output devices such as a display, a keyboard, and a mouse, and a communication circuit for communicating over a network. One or more memory devices include a computer program that may be executed by one or more processors. One or more processors may implement the functional blocks of FIG. 13 by executing the computer program. The communication unit 61 performs communication via the network by controlling the communication circuit. The communication unit 61 is also an access unit configured to be able to access the management information held by the storage apparatus 70. The communication unit 61 can access management information and acquire (download) the entirety or a part of the management information.

The input/output unit 63 controls input/output devices, receives operational input from a monitor, and presents the information generated by the generation unit 62 based on the management information to the monitor. The generation unit 62 generates information as described in FIGS. 7 to 12 based on the management information acquired by the communication unit 61 and presents the information to the monitor via the input/output unit 63. Alternatively, in response to an operation from the monitor, the generation unit 62 transmits the generated information to a PC 50 of a facility 500 specified by the monitor via the communication unit 61.

FIG. 14 is a functional block diagram of the management system 1000 according to the present embodiment. A holding unit 100 corresponds to the storage apparatus 70 and holds/stores management information. An input/output unit 102 corresponds to a PC 50, a measuring apparatus 1, and the information processing apparatus 60; adds and edits the management information; and presents and outputs information generated by a generation unit 101 to be described later based on the management information; and the like. The generation unit 101 corresponds to the information processing apparatus 60 and generates information as described in FIGS. 7 to 12 based on the management information.

<Other>

The management system 1000 of the above-described embodiment is for managing the workflow related to sterilization. However, the management system 1000 of the present invention can be made to manage workflows related to processing other than sterilization. For example, the management system 1000 can be made to manage the workflow related to cleaning. When managing cleaning, sterilization, sterilization target, sterilization apparatus, and CI in the above embodiment become cleaning, cleaning target, cleaning apparatus, and cleaning indicator, respectively. In addition, the management system 1000 can be made to manage workflows related to predetermined processing other than sterilization and cleaning, for example. In such a case, sterilization, sterilization target, sterilization apparatus, and CI in the above embodiment become predetermined processing, processing target, processing apparatus, member on which the predetermined processing is performed together with the processing target in order to determine the achievement degree of the predetermined processing, respectively. Incidentally, as described in the above embodiment, when performing predetermined processing in the processing apparatus with a set in which one or more processing targets have been packaged as a unit, one set is also one processing target.

Furthermore, the management system 1000 is not limited to the form illustrated in FIG. 2. For example, in the above embodiment, it is assumed that the storage apparatus 70 is a storage function provided by the cloud service; however, the storage apparatus 70 may be provided at the management company 600. In such a case, the storage apparatus 70 may be a storage device such as a hard disk drive (HDD) connected to the information processing apparatus 60. Alternatively, the storage apparatus 70 may be an NAS (Network Attached Storage) provided at the management company 600.

Further, in the present embodiment, the information processing apparatus 60 of the management company 600 monitors the sterilization workflows of the plurality of facilities based on the management information; however, each facility may be configured to monitor the sterilization workflow individually. In this case, the information processing apparatus 60 is arranged in the respective facilities 500 and stores, as the management information, information received from the PC 50 of the facility 500 in which it is provided. Note that, in this case, the information processing apparatus 60 cannot present information to be compared between the facilities as in the embodiment described above. However, when the facility is divided into departments or the like, information to be compared between departments can be presented. More generally, the information processing apparatus 60 manages the management information for each organization. An organization can be, for example, in units of facility such as hospitals or units of department within the same hospital. In this case, the information to be compared between the facilities of the above-described embodiment may be read as the information to be compared between the organizations. Further, when the information processing apparatus 60 is individually arranged in each facility 500, the information processing apparatus 60 can be configured to have the function of the PC 50 of the facility in which it is arranged. Further, the storage apparatus 70 may be an NAS in the facility or an HDD of the information processing apparatus 60.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-036676, filed Mar. 8, 2021 which is hereby incorporated by reference herein in its entirety.

What is claimed is:

1. A management system operable to manage a workflow including a plurality of processes for executing predetermined processing on a processing target,
the workflow including a first process and a second process executed subsequently to the first process, and
the management system comprising:
one or more processors;
one or more memories configured to store one or more programs; and
one or more storage devices configured to store, management information including information, for each of processing targets, indicating a completion timing of the first process and a start timing or a completion timing of the second process, wherein
the one or more programs, when executed by the one or more processors, cause the one or more processors to:
perform generation processing in which information for determining a temporal transition of a number of processing targets for which the first process has been completed but the second process has not been started or a number of processing targets for which the first process has been completed but the second process has not been completed is generated based on the management information.

2. The management system according to claim 1, wherein
the second process is a process in which the predetermined processing is performed on one or more processing targets by a processing apparatus,
the management information includes information indicating the start timing of the second process, and
the generation processing includes generating, based on the management information, information for determining the temporal transition of the number of processing targets for which the first process has been completed but the second process has not been started.

3. The management system according to claim 2, wherein
the processing apparatus is configured to be capable of performing the predetermined processing on the one or more processing targets at a same time,
the management information includes information for specifying the one or more processing targets on which the predetermined processing has been performed at the same time by the processing apparatus and information indicating an achievement degree of the predetermined processing by the processing apparatus, and
the generation processing includes generating, based on the management information, information for determining a relationship between a number of processing targets on which the predetermined processing has been performed at the same time by the processing apparatus and the achievement degree of the predetermined processing.

4. The management system according to claim 3, wherein the achievement degree of the predetermined processing by the processing apparatus is determined by a test paper on which the predetermined processing has been performed by the processing apparatus together with the one or more processing targets.

5. The management system according to claim 3, wherein the generation processing includes generating, for each type of the processing apparatus, information for determining the relationship between the number of the one or more processing targets for which the predetermined processing has been performed at the same time by the processing apparatus and the achievement degree of the predetermined processing.

6. The management system according to claim 3, wherein the generation processing includes generating, for each individual processing apparatus, information for determining the relationship between the number of the one or more processing targets for which the predetermined processing has been performed at the same time by the processing apparatus and the achievement degree of the predetermined processing.

7. The management system according to claim 3, wherein the generation processing includes generating, for each individual processing apparatus, information for determining a temporal transition of the achievement degree of the predetermined processing.

8. The management system according to claim 7, wherein
the generation processing includes notifying of an individual processing apparatus whose achievement degree of the predetermined processing has become lower than a first threshold in the temporal transition, and
an achievement degree of the first threshold is higher than an achievement degree of a second threshold for determining a quality of the achievement degree of the predetermined processing.

9. The management system according to claim 7, wherein the generation processing includes generating information capable of comparing the temporal transition of the achievement degree of the predetermined processing between a plurality of individual processing apparatuses.

10. The management system according to claim 3, wherein
the management information includes information indicating an organization in which the processing target is being used, and
the generation processing includes obtaining, for the one or more processing targets for which the predetermined processing has been performed at the same time by the processing apparatus, a first period, which is a sum of periods from the completion timing of the first process to the start timing of the second process and generating information for comparing, between organizations, the first periods.

11. The management system according to claim 10, wherein
the management information includes information indicating the completion timing of the second process and a completion timing of a third process executed subsequently to the second process, and
the generation processing includes obtaining, for the one or more processing targets for which the predetermined processing has been performed at the same time by the processing apparatus, a second period, which is a sum of periods from the start timing of the second process to the completion timing of the second process, and a third period, which is a sum of periods from the completion timing of the second process and the completion timing of the third process, and generating information for comparing, between organizations, second periods and third periods.

12. The management system according to claim 3, wherein
the management information includes information indicating an organization in which the processing target is being used and information for specifying a worker of a predetermined process among the plurality of processes, and
the generation processing includes generating information for comparing, between organizations, information indicating a relationship between the number of workers of the predetermined process and the achievement degree of the predetermined processing.

13. The management system according to claim 1, wherein
the predetermined processing is a sterilization,
the first process is an assembly process in which the processing target is formed by packaging one or more sterilization targets, and
the second process is a sterilization process in which one or more processing targets are sterilized by a processing apparatus.

14. The management system according to claim 1, comprises:
the one or more storage devices connected to a network; and
an information processing apparatus capable of communicating with the one or more storage devices via the network and having the one or more processors and the one or more memories.

15. The management system according to claim 14, further comprising:
one or more computers configured to transmit, to the one or more storage devices via the network, information to be added to the management information.

16. An information processing apparatus of a management system operable to manage a workflow including a plurality of processes for executing predetermined processing on a processing target,
the workflow including a first process and a second process executed subsequently to the first process, and
the information processing apparatus comprising:
one or more processors; and
one or more memories configured to store one or more programs, wherein
the one or more programs, when executed by the one or more processors, cause the one or more processors to perform generation processing including:
accessing a storage device configured to store management information, for each of processing targets, including information indicating a completion timing of the first process and a start timing or a completion timing of the second process, and
generating, based on the management information, information for determining a temporal transition of a number of processing targets for which the first process has been completed but the second process has not been started or a number of processing targets for which the first process has been completed but the second process has not been completed.

17. A non-transitory computer readable medium storing program that causes a computer having one or more processors to function as an information processing apparatus operable to manage a workflow including a plurality of processes for executing predetermined processing on a processing target,
the workflow including a first process and a second process executed subsequently to the first process, and
the program, when executed by the one or more processors, causing the computer to perform generation processing including:
accessing a storage device configured to store management information, for each of processing targets, including information indicating a completion timing of the first process and a start timing or a completion timing of the second process, and
generating, based on the management information, information for determining a temporal transition of a number of processing targets for which the first process has been completed but the second process has not been started or a number of processing targets for which the first process has been completed but the second process has not been completed.
